(19) United States
(12) Patent Application Publication
(10) Pub. No.: US 2024/0091769 A1
(43) Pub. Date: Mar. 21, 2024

(54) SYSTEM AND METHODS FOR DETECTING AN ANALYTE AND TRANSMITTING TEST ASSAY RESULTS

(71) Applicant: 3M Innovative Properties Company, St. Paul, MN (US)

(72) Inventor: Michael Wohl, Bethesda, MD (US)

(21) Appl. No.: 18/274,535

(22) PCT Filed: Jan. 28, 2022
(86) PCT No.: PCT/US2022/014388

§ 371 Date: Jul. 27, 2023

Related U.S. Application Data
(60) Provisional application No. 63/142,874, filed on Jan. 28, 2021.

(51) Int. Cl. B01L 3/00

(52) U.S. Cl. CPC B01L 3/502715; B01L 3/5023; B01L 2200/16; B01L 2300/021; B01L 2300/023; B01L 2300/0654; B01L 2300/0825

(57) ABSTRACT

Disclosed herein are test substrates, systems and methods for detecting an analyte in a sample. Test substrates of the present disclosure include a predefined pattern, wherein a first region includes a partial pattern of the predefined pattern and a second region that is incomplete and wherein the incomplete portion comprises a reagent that reacts with an analyte in a sample to produce a completed pattern, wherein the completed pattern forms a code.

### CROSS-REFERENCE TO RELATED APPLICATIONS

This application claims priority under 35 U.S.C. § 119(e) to U.S. Provisional Patent Application Ser. No. 63/142,874, filed on Jan. 28, 2021, the disclosure of which is hereby incorporated by reference in its entirety.

### BACKGROUND

The present disclosure relates generally to medicine. More particularly, the present disclosure is directed to systems and methods for detecting an analyte in a sample using a substrate having an incomplete code pattern. Upon binding of the analyte of interest in the sample, the incomplete code is converted to a completed code that can be identified and transmitted to a third party.

Test substrates are commonly used to detect the presence of analytes in samples. Test substrates can also be used to determine the concentration of analytes in samples. Test substrates can be used to detect glucose, cholesterol, enzymes, and other analytes in a sample.

A portion of a sample applied to a test substrate is transported to a test region containing a reagent that interacts with an analyte in the sample to signal the presence of the analyte. The simple operation of some test substrates allows patients without any medical training to determine the results of the test. Transmitting the signal generated by the test could be beneficial if test results are inconclusive and require interpretation by a medical professional. Transmitting the signal generated by the test could also simplify reporting to a medical agency such as the U.S. Food and Drug Administration, the U.S. Centers for Disease Control, and the European Medicines Agency.

Transmitting test results can lead to the association of the test results with a patient's identity. Disclosure of protected health information can also result in significant fines. Association of a test result to a patient's identity and disclosure of protected health information can also be used for malicious purposes. Thus, systems and methods that encrypt test results provides security protection of protected health information and allows for the secure transmission of test results. Encryption of the test results would protect a patient's protected health information including test results in the event of transmission of the wrong patient's information.

Accordingly, there exists a need for systems and methods for detecting an analyte in a sample and transmitting the results.

### BRIEF DESCRIPTION

The present disclosure generally relates to medicine. More particularly, the present disclosure is directed to systems and methods for detecting an analyte in a sample using a substrate having an incomplete code pattern. Upon binding of the analyte of interest in the sample, the incomplete code is converted to a completed code that can be identified and transmitted to a third party.

In one aspect, the present disclosure is directed to a test substrate for detecting an analyte in a sample comprising: a predefined pattern comprising a first region comprising a partial pattern of the predefined pattern and a second region, wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code.

In one aspect, the present disclosure is directed to a system for detecting an analyte in a sample obtained from a subject and transmitting a test result, the system comprising: an analytical assay that comprises: a test substrate, the test substrate comprising: a predefined pattern comprising a first region comprising a partial pattern of the predefined pattern and a second region, wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code; and a processor and a memory, wherein the processor can receive an image profile associated with the test substrate, process the image, and transmit the image.

In one aspect, the present disclosure is directed to a computer-implemented method for detecting an analyte in a sample and generating a scannable pattern, the method performed by an analysis computing device comprising a processor and a memory, the method comprising: receiving an image profile associated with a test substrate profile, wherein the test substrate profile defines a layout for a test substrate, the test substrate comprising: a predefined pattern comprising a first region comprising a partial pattern of the predefined pattern and a second region, wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code; receiving an image of the code; and processing the image of the code.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is an illustration depicting results of contacting a test substrate of an exemplary embodiment with a sample without an analyte and contacting a test substrate with an analyte. For illustrative purposes the upper left quadrant of the Test Substrate with Incomplete Pattern is visible in the figure to illustrate the predefined pattern. When the sample with analyte is contacted with the test substrate, the analyte (if present in the sample) interacts with a reagent in the upper left quadrant to produce a signal and result in a complete pattern. The complete pattern produces a code.

FIG. 2 is an illustration of a test substrate such as used in a lateral flow assay or vertical flow assay in which a sample containing an analyte is contacted with the test substrate at a sample application area and flows through a conjugate pad and then to the reagent test region (shown “blank” and as dashed vertical lines). A signal is produced at the test region to complete the pattern and reveal a completed code.

FIG. 3 is an illustration as described in FIG. 2, but depicting a signal generated in the reagent region (e.g., the control signal line) that can represent a positive control to indicate that the test assay worked properly and that the sample did not contain the analyte of interest. A signal generated by the control completes the pattern at the control line and reveals a code. Interpretation of the resulting code indicates that the control was detected and that no analyte was present in the sample.

FIG. 4 is an illustration depicting an exemplary embodiment using a QR code-type of code. A sample with an analyte is applied to a sample pad and flows through a conjugate pad. When the analyte reaches the test region, a signal produces a pattern to complete the pattern and reveal a QR code.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is directed to systems and methods for detecting an analyte in a sample using a substrate having an incomplete pattern. Upon completion of the pattern a code is produced that provides the results of the test assay.

In one aspect, the present disclosure is directed to a test substrate for detecting an analyte in a sample. The test substrate includes: a predefined pattern. The predefined pattern includes at least a first region and a second region. The first region includes a partial pattern of the predefined pattern. The second region of the predefined pattern is incomplete. In other words, the second region refers to the missing portion of the predefined pattern. The second region (also referred to herein as the “test region”) includes a reagent. A signal produced in the second region results in the production of a pattern. The combination of the partial pattern of the first region and the pattern produced by the signal in the second region completes the predefined pattern to complete the pattern and produce a code. It should be understood that a “complete” pattern can be a partial pattern and still produce a code. An example of this embodiment is that a sample that does not contain an analyte, but does include a control molecule, can result in the production of a signal from a control line, but not in the test line, for example. The resultant code would reveal that the control molecule was detected, but no analyte was detected. Another example of this embodiment would be a multiplexing assay, where a signal is produced to detect an first analyte and a control molecule, but not a second analyte. Thus, a “complete” code would be produced even though the test line to detect the second analyte remained blank (or missing). It should also be understood that a “complete” pattern can be a whole pattern to produce a code. An example of this embodiment is that a sample with an analyte and a control molecule will produce signals in both the test line and the control line. The resultant code would reveal that both the analyte and the control molecule were detected in the sample. Another example of this embodiment would be a multiplexing assay, where a signal is produced to detect an first analyte, a second analyte, and a control molecule. Thus, a “complete” code would be produced from signals generated from the first analyte test line, the second analyte test line, and the control test line. It should also be understood that the first region including the partial pattern of the predefined pattern can define a code, and thus, provide information even in the absence of a signal produced in the second region (by, for example, a test line and/or a control line). For example, if a signal for the test line and/or the control line fails to be generated, the partial pattern of the first region can be analyzed to provide feedback that the analyte was not detected and/or that the control molecule was not detected.

The test substrate is suitably made with synthetic fibers, natural fibers, and combinations thereof. Fibers used to form the substrate include hydrophobic fibers, hydrophilic fibers, and combinations thereof. Hydrophobic fibers include, for example, polylactones, poly(caprolactone), poly (L-lactic acid), poly (glycolic acid), similar co-polymers poly(alkyl acrylate), polybutadiene, polyethylene, polystyrene, polyacrylonitrile, poly ethylene (terephthalate), polysulfone, polycarbonate, poly(vinyl chloride), and combinations thereof. Hydrophilic fibers include, for example, linear poly(ethylenimine), cellulose, cellulose acetate and other grafted cellulosics, poly (hydroxyethylmethacrylate), poly (ethyleneoxide), polyvinylpyrrolidone, poly(acrylic acid), poly(ethylene glycol), poly(vinyl alcohol), poly (vinyl acetate), poly(acrylamide), proteins, poly (vinyl pyrrolidone), poly(styrene sulfonate), and combinations thereof. Other suitable fiber materials include, for example, acrylonitrile/butadiene copolymer, cellulose, cellulose acetate, chitosan, collagen, DNA, fibrinogen, fibronectin, nylon, poly(acrylic acid), poly(chloro styrene), poly(dimethyl siloxane), poly(ether imide), poly(ether sulfone), poly(ethyl acrylate), poly(ethyl vinyl acetate), poly(ethyl-co-vinyl acetate), poly(ethylene oxide), poly(ethylene terephthalate), poly(lactic acid-co-glycolic acid), poly(methacrylic acid) salt, poly(methyl methacrylate), poly(methyl styrene), poly(styrene sulfonic acid) salt, poly(styrene sulfonyl fluoride), poly(styrene-co-acrylonitrile), poly(styrene-co-butadiene), poly(styrene-co-divinyl benzene), poly(vinyl acetate), poly(vinyl alcohol), poly(vinyl chloride), poly(vinylidene fluoride), polyacrylamide, polyacrylonitrile, polyamic acid (PAA), polyamide, polyaniline, polybenzimidazole, polycaprolactone, polycarbonate, polydimethylsiloxane-co-polyethyleneoxide, polyetheretherketone, polyethylene, polyethyleneimine, polyimide, polyisoprene, polylactide, polypropylene, polystyrene, polysulfone, polyurethane, polyvinylpyrrolidone, proteins, SEBS copolymer, silk, and styrene/isoprene copolymer. Polymer blends such as, for example, poly(vinylidene fluoride)-blend-poly(methyl methacrylate), polystyrene-blend-poly (vinylmethylether), poly(methyl methacrylate)-blend-poly(ethyleneoxide), poly(hydroxypropyl methacrylate)-blend poly(vinylpyrrolidone), poly(hydroxybutyrate)-blend-poly(ethylene oxide), protein blend-polyethyleneoxide, polylactide-blend-polyvinylpyrrolidone, polystyrene-blend-polyester, poly ester-blend-poly(hyroxyethyl methacrylate), poly (ethylene oxide)-blend poly(methyl methacrylate), poly(hydroxy styrene)-blend-poly(ethylene oxide).

Fluid sample is transported through the test substrate to the region of the substrate where the predefined pattern is incomplete at the second region of the predefined pattern. Upon reaction between the analyte and a reagent, the incomplete pattern is filled in or completed by the production of a signal. A combination of the partial pattern of the first region of the predefined pattern and the signal generated in the second region, the incomplete pattern is completed to reveal a code. For example, the testing reagent located in the second region of the code reacts with the analyte to produce a color change to produce a completed code. Thus, the incomplete region of the code is filled in to complete the pattern. The signal created in the second region can depend on the concentration of analyte in the sample and allows for determining the presence of the analyte in the sample, the concentration of the analyte in the sample, and combinations thereof.

Suitable patterns to make a transmittable code include barcodes such as PDF417 codes, quick response (“QR”) codes, data matrix codes, GS1 composite code, MaxiCode, Aztec code, dot code, UPC-A codes, UPC-E codes, EAN-13 codes, industrial 2 of 5, interleaved 2 of 5, codabar, code 11, code 39, code 93, code 128, DataBar, MSI code, Patch code, USPS intelligent mail code, Postnet code, Planet code, Australian Post code, UK Royal mail code, shapes, words, numbers, characters, and combinations thereof. The patterns making the code can be one-dimensional codes, two-dimensional codes, and three-dimensional codes.

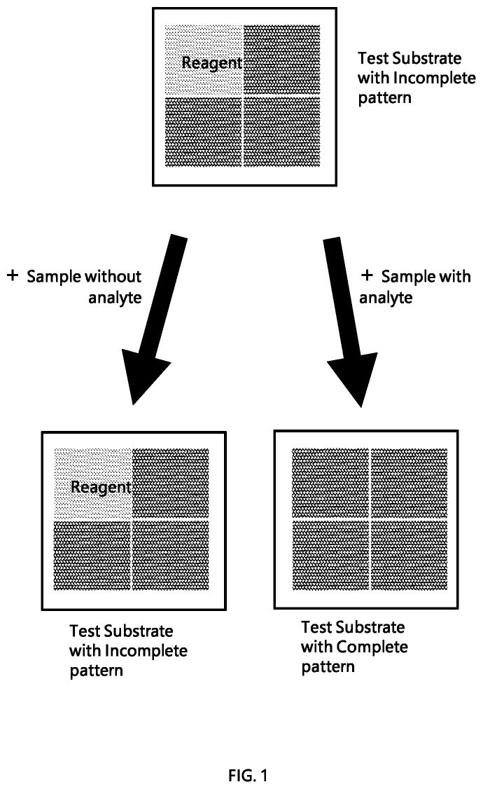

With reference to FIG. 1, a test substrate has four quadrants. In the top left quadrant is the “incomplete portion” of the pattern where the reagent is located. The partial pattern is created by the upper right quadrant, the lower left quadrant, and the lower right quadrant. A sample containing an analyte is applied to the test substrate. Analyte in the sample interacts with the reagent in the upper right quadrant to produce a signal that completes the pattern and reveals the code.

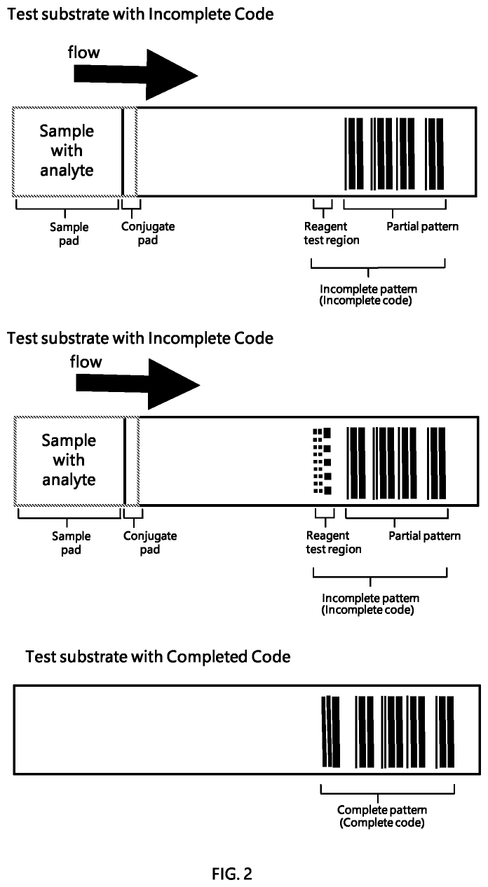

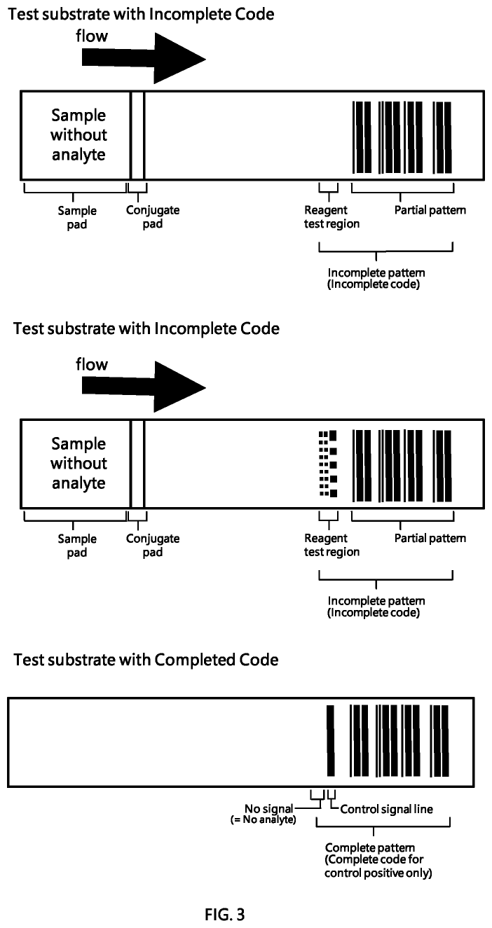

FIG. 2 illustrates a barcode-like embodiment. A test substrate such as that used in a lateral flow assay and a vertical flow assay includes a partial barcode (as indicated by solid lines of different thickness). A sample is applied to a sample region and fluid flows to a conjugate region where an analyte in the sample reacts with a labeled binding partner (such as an antibody). The analyte that is now bound with its labeled binding partner flows into the test region where a second binding partner that is immobilized in the test region captures the analyte to generate a signal. The signal results in filling in the pattern to reveal the barcode. It should be recognized that the test region can also include a control line that includes affinity ligands that show whether the sample has flowed through and the bio-molecules in the conjugate pad are active. In the absence of the analyte, no signal will be generated in the test region and the incomplete portion of the barcode will be absent. It should be understood that a signal indicating detection of the control will fill in a portion of the pattern and reveal the code. Thus, the code can indicate both a positive result where a signal is created by the analyte being detected and a signal that is created by the control. A code can also be revealed that indicates only the signal generated by the control as depicted in FIG. 3.

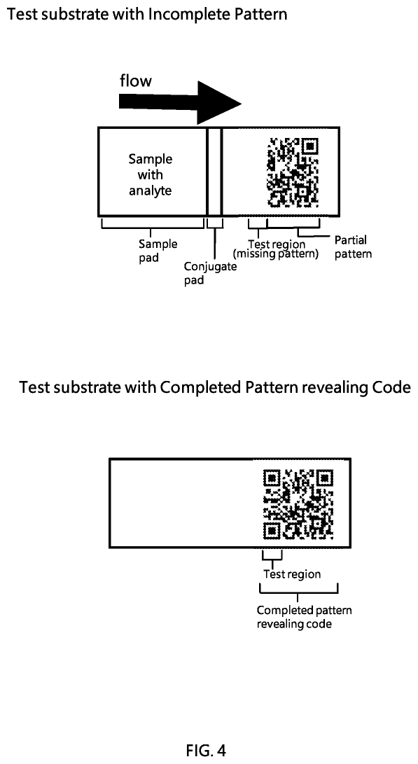

FIG. 4 illustrates a QR code-like embodiment. A sample is applied to a region of the test substrate. When an analyte reaches the test region a signal is produced that completes the pattern to form the QR code.

The test substrate is particularly suitable for use in lateral flow assays and vertical flow assays. As known in the art, in lateral flow assays and vertical flow assays, a sample pad acts as a sponge and holds a sample fluid. Once soaked, the fluid flows to a conjugate pad that contains a labeled binding partner that specifically binds the analyte. The conjugate pad also includes all of the reagents required for a chemical reaction between the analyte and the binding partner to form a complex. The complex passes through the pad and flows to the test region and control region. The test line shows a signal, often a color as in pregnancy tests. The control line contains affinity ligands that show whether the sample has flowed through and that the molecules in the conjugate pad are active. After passing the test region and the control region, the fluid enters the final porous material. Signals created in the test region and control region will fill in a portion of the pattern to generate a code.

The test substrate of the present disclosure is suitable for use in both sandwich assays and competitive assays formats.

The codes can be used to transfer data. The data can be a positive result, a negative result, a control result, and the like. The portion of the code can include data and/or information such as the type of analytical assay, lot number(s) of the assay, the reagent(s) used in the substrate, lot numbers of the reagent(s) used in the substrate, date(s) of the assay such as expiration dates and production dates, batch numbers, test substrate expiration date, electronic tag cryptogram, a reference value of a clinical index, and test substrate manufacturer information, and identity information of a tested object, tester cryptogram, a sample name, a sample sequence number, test date and combinations thereof.

The code can be scanned using a variety of devices. Suitable devices The same devices used to scan the code can also be used to transmit the code. For example, a cameral of a mobile phone can be used to scan and/or take a photograph of the code after the analytical assay is complete. A mobile app on a mobile phone can be used to transmit the code. The transmitted code is received by a terminal as described herein.

The test substrate can be an immunochromatographic test substrate, a chemiluminescence test substrate, a colloidal gold test substrate, a colloidal selenium test substrate, a quantum dot test substrate, an upconversion phosphorescence test substrate, a nano rare earth fluorescent complex test substrate, a temporal resolution chromatography test substrate, and other test substrates.

The test substrate can also be a multi-layer substrate. The multi-layer test substrate is suitably made of a material having high surface area for collecting the analyte to be detected. The multi-layer substrate can also include protective layers. The protective layers and test layer can be made of different materials designed for the specific purposes of the layer (e.g., protection and sample collection).

The test substrate is processed to detect an analyte of interest on the test substrate. For example, a test substrate can be analyzed by adding a reaction solution (e.g., buffer and/or water) to the test substrate that results in a colorimetric reaction indicating the presence or absence of the analyte by completion of the pattern to form a code. In another embodiment, the test substrate is placed in a reaction solution (e.g., buffer and/or water). If the analyte is present, completion of the pattern forms a code.

Suitable reagents include salts, pH buffers, preservatives, capture reagents, analysis reagents, detection reagents, adhesives, fragrances, odor absorbing compounds, preservatives, RNAse inhibitors, protease inhibitors, and nucleic acids, including deoxyribonucleic acid, ribonucleic acid, and nucleotide analogs and the like. Other suitable reagents include capture reagents, for example, antibodies that specifically binds an analyte of interest, a ligand that specifically binds an analyte of interest such as, for example, surface molecules, such as sugars, glycoproteins, and the like. The reagents can be covalently and/or noncovalently coupled to the test substrate directly or by a linker. Any suitable linker can be used such as, for example, organic molecules such as a polymer or copolymer (e.g., a substituted or unsubstituted polyalkylene glycol, such as polyethylene glycol), and/or biological molecules such as bovine serum albumin. When the binding of the reagent is intended to be non-covalent, the reagent can be, for example, impregnated in the test substrate by applying a solution containing the reagent to the test substrate and subsequently evaporating the solvent.

The test substrate can be used to capture any analyte of interest. Suitable analytes can be viruses, bacteria, nucleic acids (e.g., DNA and/or RNA), proteins, chemicals, and combinations thereof. As described herein, pore size of the test substrate is designed such that the analyte of interest is captured by the test substrate.

Suitable samples include liquid samples such as body fluids. Body fluids include blood (e.g., whole blood, plasma, and serum), urine, sputum, saliva, expelled breath (cough, blowing, etc.), cerebrospinal fluid, tissue homogenates, liquid tissue extracts, and the like. The sample can be used to test for infectious disease, cancers, diabetes, cardiovascular diseases, tumors, hormones, autoimmune disease, and the like. The test substrate can also be used in assays for drug detection, food detection, biohazard detection, explosives detection, environmental pollution detection, pesticide residue detection, biological contamination detection, biological agent detection, veterinary detection, and the like.

In another aspect, the present disclosure is directed to a system for analyzing a sample and reporting results of the analysis. The system comprising: an analytical assay that comprises: a test substrate, the test substrate comprising: a predefined pattern comprising a first region comprising a partial pattern of the predefined pattern and a second region, wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code; and a processor and a memory, wherein the processor can receive an image profile associated with the test substrate, process the image, and transmit the image. The system includes the test substrate as described herein, components for reading the pattern, the code, and combinations thereof. The system also includes components for transmitting the code. The system can include components for encrypting the code. The system also includes components for receiving the code. The system also includes components for interpreting the code. The system also includes components for displaying the code. The system also includes a processor. The system also includes component for data management. The system also includes components for feedback. The feedback can be provided to a medical professional, an agency, and the patient. The feedback can be the results of the test such as diagnosis of a disease, a concentration of an analyte in a sample, and the like. While the code includes test result information, the meaning of the code (such as a barcode) may not be readily apparent to one observing the code.

The system includes a wireless communication module connected with a data processor, and the power supply that is configured to supply power to the wireless communication module; and the test substrate. The wireless communication module includes a wireless network system, that is in communication with a signal transmission end of the wireless communication module and a remote server. The data processor is configured to identify the optical signals of the test line and the control line. The data processor can calculate the concentration of the detected analyte and perform technical analysis according to the standard curve of the detected analyte stored on the test substrate. The code is transmitted by a reading and writing module, and thus, transmit a test result to the output display, wherein the test result and the related information of the code are sent to a remote server of the wireless network system via the wireless communication module for data management and information consultation feedback.

The system can determine the amount or concentration of the analyte in the sample by obtaining an optical density of the signal generated at the test line and an optical density of the control signal.

The system can include a portable instrument including wireless communication products having information transmitting and receiving functions. Suitable instruments include, for example, mobile phones, tablet computers, personal digital assistants, a computer, and combinations thereof.

In another aspect, the present disclosure is directed to a computer-implemented method for detecting an analyte in a sample and generating a scannable pattern, the method performed by an analysis computing device comprising a processor and a memory, the method comprising: receiving an image profile associated with a test substrate profile, wherein the test substrate profile defines a layout for a test substrate, the test substrate comprising: a predefined pattern comprising a first region comprising a partial pattern of the predefined pattern and a second region, wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code; receiving an image of the code; and processing the image of the code.

The test substrates disclosed herein allow for the detection of an analyte in a sample similar to other known test substrates. Unlike known test substrates, the signal generated in the test assay is used to complete a pattern included on the test substrate. Advantageously, the completed pattern results in the formation of a code. In addition to the test assay results, the code contains additional information. Transmission of the code results in the reporting of the test assay results in combination with the additional data contained in the code. The code can be transmitted by a patient using an at-home test and received by medical professionals and regulatory agencies.

Additionally, the test substrates of the present disclosure are universally applicable to a variety of test platforms such as lateral flow assays, vertical flow assays. Because the test results form part of pattern that results in the formation of a code, the code and all of the information provided in the code can be encrypted. This advantageously allows for the transmission of information such as protected health information and allows for its secure transmission.

What is claimed is:

1. A test substrate for detecting an analyte in a sample comprising:
a predefined pattern comprising
a first region comprising a partial pattern of the predefined pattern and a second region,
wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code.

2. The substrate of claim 1, wherein the code is selected from the group consisting of a PDF417 code, a quick response (“QR”) code, a data matrix code, a GS1 composite code, a MaxiCode, an Aztec code, a dot code, an UPC-A code, an UPC-E code, an EAN-13 code, an industrial 2 of 5 code, an interleaved 2 of 5 code, a codabar, a code 11, a code 39, a code 93, a code 128, a DataBar, a MSI code, a Patch code, a USPS intelligent mail code, a Postnet code, a Planet code, an Australian Post code, a UK Royal mail code, a shape, a word, a number, a character, and combinations thereof.

3. The substrate of claim 1, wherein the code is a one-dimensional code, a two-dimensional code, and a three-dimensional code.

4. The substrate of claim 1 further comprising a sample application region.

5. The substrate of claim 1 further comprising a conjugate region.

6. The substrate of claim 1 further comprising a wicking region.

7. A method of analyzing a sample obtained from a subject, the method comprising contacting the sample with the substrate of claim 1.

8. The method of claim 7, wherein the sample is analyzed for presence of an analyte in the sample.

9. The method of claim 7, wherein the sample is analyzed for an amount of an analyte in the sample.

10. The method of claim 7, wherein the analysis is a competitive assay.

11. The method of claim 7, wherein the analysis is a sandwich assay.

12. The method of claim 7, further comprising scanning the code.

13. The method of claim 7, further comprising transmitting the code.

14. The method of claim 7, further comprising transmitting using a mobile phone.

15. A system for detecting an analyte in a sample obtained from a subject and transmitting a test result, the system comprising:
an analytical assay that comprises: a test substrate, the test substrate comprising:
a predefined pattern comprising
a first region comprising a partial pattern of the predefined pattern and
a second region,
wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code; and
a processor and a memory, wherein the processor can receive an image profile associated with the test substrate, process the image, and transmit the image.

16. A computer-implemented method for detecting an analyte in a sample and generating a scannable pattern, the method performed by an analysis computing device comprising a processor and a memory, the method comprising:
receiving an image profile associated with a test substrate profile,
wherein the test substrate profile defines a layout for a test substrate, the test substrate comprising:
a predefined pattern comprising
a first region comprising a partial pattern of the predefined pattern and
a second region,
wherein the second region of the predefined pattern is incomplete and comprises a reagent, where upon binding to an analyte produces a signal resulting in the production of a pattern in the second region, wherein a combination of the partial pattern of the first region and the pattern in the second region completes the predefined pattern to produce a code;
receiving an image of the code; and
processing the image of the code.

17. The computer-implemented method of claim 16, wherein the analysis computing device is in communication with a camera configured to capture the image of the first test substrate.

18. The computer-implemented method of claim 17, further comprising:
receiving a first image of the test substrate before the sample is applied to the test substrate; and
defining the image profile of the predefined pattern before the sample is applied to the test substrate.

19. The computer-implemented method of claim 17, further comprising:
receiving a second image of the test substrate after the sample is applied to the test substrate; and
defining the image profile of the predefined pattern after the sample is applied to the test substrate.
